(19) United States
(12) Patent Application Publication
(10) Pub. No.: US 2009/0164023 A1
(43) Pub. Date: Jun. 25, 2009

(54) ASSEMBLY COMPRISING COMPOSITE MATERIALS FOR BEARING SURFACES AND USES THEREOF IN RECONSTRUCTIVE OR ARTIFICIAL JOINTS

(75) Inventor: John Neil Devine, Lancashire (GB)

Correspondence Address: MICHAEL BEST & FRIEDRICH LLP, 100 E WISCONSIN AVENUE, Suite 3300, MILWAUKEE, WI, 53202, US

(73) Assignee: INVIBIO LIMITED, Lancashire (GB)

(21) Appl. No.: 12/281,223

(22) PCT Filed: Feb. 28, 2007
(86) PCT No.: PCT/GB07/00689

§ 371 Date: Oct. 6, 2008

(30) Foreign Application Priority Data
Mar. 1, 2006 (GB) 0604061.2

(51) Int. Cl. A61F 2/32; A61F 2/30; A61L 27/44

(52) U.S. Cl. 623/22.15; 623/18.11; 29/428

(57) ABSTRACT

An assembly, for example for a reconstructive joint of the human body, comprises first and second parts which bear against one another. The first and second parts may both comprise a first polymeric material which is preferably polyetheretherketone in combination with carbon fibre.

This invention relates to polymeric materials and particularly, although not exclusively, relates to the use of such materials in assemblies comprising first and second parts which bear against one another. Preferred embodiments relate to the use of composite materials for bearing surfaces, for example for reconstructive joints (or other parts) of human bodies.

A wide range of materials has been proposed for use in reconstructive or artificial joints (or other parts) of human bodies, for example for joints or bearing surfaces in the spine; for shoulder or finger joints; and for partial or total hip or knee replacements.

Tribiology International Vol. 31, No. 11, pp 661-667, 1998 (Wang) describes the success of total hip arthroplasty in the second half of the 20^{th} century as owing greatly to the use of ultra-high molecular weight polyethylene as a bearing surface for the acetabular component. Excellent wear is acknowledged when a polyethylene bearing surface is coupled with a metal or ceramic femoral head. However, a problem is acknowledged in that the debris produced by wear of polyethylene may cause adverse biological reaction, leading to bone loss or osteolysis, and, subsequently, the need to undertake revision surgery.

Metal-on-metal articulation joints have been proposed and used with mixed results. Some metal implants may fail in a relatively short time whilst some will last much longer. Such inconsistent performance is, of course, unacceptable. It may stem from difficulties in controlling manufacturing tolerance of a metal-on-metal implant such as clearance, sphericity, surface finish or the quality of the alloy itself.

Ceramic-on-ceramic joints have been proposed but these require even higher manufacturing precision than metal-on-metal joints because of the inherent brittleness of the ceramic.

Thus metal-on-metal and ceramic-on-ceramic joints are much less forgiving in the design and manufacturing areas and more sensitive to surgical techniques compared to polyethylene/metal joints.

Another problem with known materials is the tendency for them to exhibit increased wear as the load on them increases. If, in a joint, there is anything other than a perfect fit between two bearing parts, the joint may wear more quickly than expected (due to the increased load), leading to premature failure of the joint.

It is an object of the present invention to address the aforementioned problems.

According to a first aspect of the invention, there is provided an assembly comprising:

(a) a first part which comprises a first composite material which includes a first polymeric material and carbon fibre, wherein said first polymeric material includes a repeat unit of formula

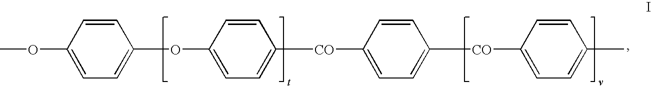

and;
(b) a second part which comprises a second composite material which includes a second polymeric material and carbon fibre, wherein said second polymeric material includes a repeat unit of formula

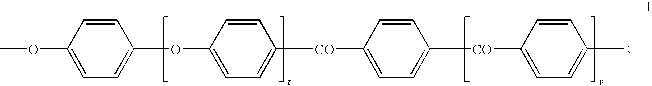

wherein said first and second parts bear against one another.

Said first and second parts may bear against one another so that, in use, one or both of the parts may have a tendency to wear and/or produce wear debris by virtue of contact between the parts. Advantageously, however, the materials from which the first and second parts are made may be such that the amount of wear debris produced and the rate of wear is significantly less than for corresponding parts made from other polymeric materials such as acetal or ultra-high molecular weight polyethylene.

Preferably, a bearing surface of said first part which comprises said first composite material contacts a bearing surface of said second part which comprises said second composite material. Thus, in the assembly, a bearing surface which comprise said first composite material suitably contacts a bearing surface which comprises said second composite material.

In the assembly, said first part and said second part are preferably movable relative to one another. For example, a bearing surface of one of the parts may be arranged to slide over a bearing surface of the other part. Said first and second parts may be pivotable relative to one another.

Said first and second parts are preferably lubricated in use. For example they may be lubricated by synovial fluid when used in a human body; or lubricated by a lubrication fluid such as an oil, when used in other applications. Many different types of assemblies comprising first and second parts as described may be provided. Preferably, said assembly is for implantation in a human body, suitably to replace a structural element of the human body. Said assembly may be for use in or around the spine, for example in spinal non-fusion technologies; or for use in artificial joints, for example in fingers, hips, knees, shoulders, elbows, toes and ankles.

One of said first or second parts of the assembly may comprise a male element and the other of said first or second parts may comprise a female element wherein said male and female elements bear against one another, suitably with said bearing surfaces which comprise said first composite material and said second composite material in contact, and said male element is pivotable relative to the female element.

Said first part may be made substantially entirely from said first composite material. Alternatively, a first part may comprise a material other than said first composite material but a bearing surface of such a first part may be defined by said first composite material. Such a bearing surface may be defined by capping or coating, or otherwise providing, a layer of first composite material on a precursor of said first part for defining said first part. For example, said first part may comprise a metal or ceramic part (e.g. a femoral head) which is capped with said first composite material or the first part may comprise bone (i.e. the natural bearing material) wherein a bearing surface is capped or otherwise resurfaced with said first composite material.

Said second part may be made substantially entirely from said second composite material. Alternatively, a second part may comprise a material other than said second composite material but a bearing surface of such a second part may be defined by said second composite material. Such a bearing surface may be defined by capping or coating, or otherwise providing, a layer of second composite material on a precursor of said second part for defining said second part. For example, said second part may comprise a metal or ceramic part (e.g. a femoral head) which is capped with said second composite material or the second part may comprise bone (i.e. the natural bearing material) wherein a bearing surface is capped or otherwise resurfaced with said second composite material.

One of said first or second parts of the assembly may define a head and the other part may define a socket within which the head is pivotable.

In a preferred embodiment, said assembly may be for a hip replacement. It may comprise a femoral head and an acetabular component. Bearing surfaces which contact one another suitably are defined by said first composite material and said second composite material.

Said first polymeric material preferably includes a said repeat unit I wherein t and v independently represent 0 or 1. Preferred polymeric materials have a said repeat unit wherein either t=1 or v=0; t=0 and v=0; or t=0 and v=1. More preferred have t=1 and v=0; or t=0 and v=0. The most preferred has t=1 and v=0.

Said first polymeric material preferably includes at least 60 mole %, more preferably at least 90 mole % of repeat units of formula I. Preferably, said first polymeric material consists essentially of repeat units of formula I. Preferably, said first polymeric material includes a single type of repeat unit of formula I.

In preferred embodiments, said first polymeric material is selected from polyetheretherketone, polyetherketone and polyetherketoneketone. In a more preferred embodiment, said first polymeric material is selected from polyetherketone and polyetheretherketone. In an especially preferred embodiment, said first polymeric material is polyetheretherketone.

Thus, preferably, said first polymeric material consists essentially of a repeat unit of formula I wherein t=1 and v=0.

Said first polymeric material suitably has a melt viscosity (MV) of at least 0.06 kNsm⁻², preferably has a MV of at least 0.09 kNsm⁻², more preferably at least 0.12 kNsm⁻², especially at least 0.15 kNsm⁻².

MV is suitably measured using capillary rheometry operating at 400° C. at a shear rate of 1000 s⁻¹ using a tungsten carbide die, 0.5×3.175 mm.

Said first polymeric material may have a MV of less than 1.00 kNsm⁻², preferably less than 0.5 kNsm⁻².

Said first polymeric material may have a MV in the range 0.09 to 0.5 kNsm⁻², preferably in the range 0.14 to 0.5 kNsm⁻².

Said first composite material may have an MV in the range 0.5 to 1.0 kNsm⁻², preferably in the range 0.7 to 1.0 kNsm⁻². MV may be measured by capillary rheometry.

Said first polymeric material may have a tensile strength, measured in accordance with ASTM D790 of at least 40 MPa, preferably at least 60 MPa, more preferably at least 80 MPa. The tensile strength is preferably in the range 80-110 MPa, more preferably in the range 80-100 MPa.

Said first composite material may have a tensile strength, measured in accordance with ASTM D790 of greater than 100 MPa, preferably of greater than 120 MPa.

Said first polymeric material may have a flexural strength, measured in accordance with ASTM D790 of at least 145 MPa. The flexural strength is preferably in the range 145-180 MPa, more preferably in the range 145-165 MPa.

Said first composite material may have a flexural strength, measured in accordance with ASTM D790, of at least 200 MPa.

Said first polymeric material may have a flexural modulus, measured in accordance with ASTM D790, of at least 2 GPa, preferably at least 3 GPa, more preferably at least 3.5 GPa. The flexural modulus is preferably in the range 3.5-4.5 GPa, more preferably in the range 3.5-4.1 GPa.

Said first composite material may have a flexural modulus, measured in accordance with ASTM D790, of at least 7 GPa.

Advantageously, the first polymeric material and said carbon fibre may be selected to tailor the properties of the first composite material. For example, the flexural modulus may be tailored to that of cortical bone (approximately 18 GPa).

Said second polymeric material preferably includes a said repeat unit I wherein t and v independently represent 0 or 1. Preferred polymeric materials have a said repeat unit wherein either t=1 or v=0; t=0 and v=0; or t=0 and v=1. More preferred have t=1 and v=0; or t=0 and v=0. The most preferred has t=1 and v=0.

Said second polymeric material preferably includes at least 60 mole %, more preferably 90 mole % of repeat units of formula I. Preferably, said second polymeric material consists essentially of repeat units of formula I. Preferably, said second polymeric material includes a single type of repeat unit of formula I.

In preferred embodiments, said second polymeric material is selected from polyetheretherketone, polyetherketone and polyetherketoneketone. In a more preferred embodiment, said second polymeric material is selected from polyetherketone and polyetheretherketone. In an especially preferred embodiment, said second polymeric material is polyetheretherketone.

Thus, preferably, said second polymeric material consists essentially of a repeat unit of formula I wherein t=1 and v=0.

Said second polymeric material suitably has a melt viscosity (MV) of at least 0.06 kNsm⁻², preferably has a MV of at least 0.09 kNsm⁻², more preferably at least 0.12 kNsm⁻², especially at least 0.15 kNsm⁻².

MV is suitably measured using capillary rheometry operating at 400° C. at a shear rate of 1000 s⁻¹ using a tungsten carbide die, 0.5×3.175 mm.

Said second polymeric material may have a MV of less than 1.00 kNsm⁻², preferably less than 0.5 kNsm⁻².

Said second polymeric material may have a MV in the range 0.09 to 0.5 kNsm⁻², preferably in the range 0.14 to 0.5 kNsm⁻².

Said second composite material may have an MV in the range 0.5 to 1.0 kNsm⁻², preferably in the range 0.7 to 1.0 kNsm⁻².

Said second polymeric material may have a tensile strength, measured in accordance with ASTM D790 of at least 40 MPa, preferably at least 60 MPa, more preferably at least 80 MPa. The tensile strength is preferably in the range 80-110 MPa, more preferably in the range 80-100 MPa.

Said second composite material may have a tensile strength, measured in accordance with ASTM D790 of greater than 100 MPa, preferably of greater than 120 MPa.

Said second polymeric material may have a flexural strength, measured in accordance with ASTM D790 of at least 145 MPa. The flexural strength is preferably in the range 145-180 MPa, more preferably in the range 145-165 MPa.

Said second composite material may have a flexural strength, measured in accordance with ASTM D790, of at least 200 MPa.

Said second polymeric material may have a flexural modulus, measured in accordance with ASTM D790, of at least 2 GPa, preferably at least 3 GPa, more preferably at least 3.5 GPa. The flexural modulus is preferably in the range 3.5-4.5 GPa, more preferably in the range 3.5-4.1 GPa.

Said second composite material may have a flexural modulus, measured in accordance with ASTM D790, of at least 7 GPa.

Advantageously, the second polymeric material and said carbon fibre may be selected to tailor the properties of the second composite material. For example, the flexural modulus may be tailored to that of cortical bone (approximately 18 GPa).

Preferably, said first polymeric material and said second polymeric material are the same.

Said carbon fibre may be of any suitable type. Said carbon fibre may be PAN-based or pitch based.

Suitable PAN-based fibres may have a fibre density in the range 1.7 to 1.85 g.cm⁻³, a tensile strength of greater than 2900 MPa, a tensile modulus in the range 230-250 GPa a bulk density of greater than 350 g/l.

Suitable pitch-based carbon fibres may have a fibre density in the range 1.2-2 g.cm⁻³, a tensile strength in the range 400-600 MPa and a Young's Modulus of 30-50 GPa.

Said carbon fibre may comprise milled forms, for example having average lengths in the range 200-1600 μm. Alternatively, the carbon fibres may be in chopped lengths for example having average lengths in the range 3 to 30 mm. In a further alternative, endless carbon fibres may be present in the first and/or second composite materials. Such endless materials may comprise 6000 or 12000 filament tows.

The carbon fibres may incorporate additives or a finish as is conventional for such materials to improve compatibility of the fibres with the first and second polymeric materials.

Preferably, said first part comprises a first composite material comprising said first polymeric material and PAN-based carbon fibres. Preferably PAN-based fibres make up at least 50 wt %, at least 75 wt %, at least 90 wt %, at least 95 wt %, especially about 100 wt % of the carbon fibre of the first composite material. Preferably, said second part comprises said second composite material comprising said second polymeric material and PAN-based carbon fibres. Preferably PAN-based fibres make up at least 50 wt %, at least 75 wt %, at least 90 wt %, at least 95 wt %, especially about 100 wt % of the carbon fibre of the second composite material.

Said first composite material suitably includes at least 30 wt %, preferably at least 45 wt %, more preferably at least 60 wt %, especially at least 65 wt % of said first polymeric material. Said composite material may include up to 70 wt %, up to 55 wt %, up to 40 wt %, up to 35 wt % of carbon fibres. Said first composite material may include 30 to 70 wt % of said first polymeric material and 30 to 70 wt % of carbon fibre. In a preferred embodiment said first composite material comprises 60 to 80 wt % of polymeric material of formula I, preferably of formula I wherein t=1 and v=0, and 20 to 40 wt % of carbon fibre.

Said first composite material may include one or more further components. It may include up to 15 wt %, preferably up to 10 wt % of other components. An example of another component is an X-ray contrast material for example barium sulphate.

Said first composite material preferably includes only a single type of first polymeric material of formula I. It may also include only a single type of carbon fibre—e.g. only PAN-based; or only pitch-based, but not a mixture of two types.

Said carbon fibre of the second composite material may independently have any features of the carbon fibre of the first composite material.

Said second composite material suitably includes at least 30 wt %, preferably at least 45 wt %, more preferably at least 60 wt %, especially at least 65 wt %, of said second polymeric material. Said composite material may include up to 70 wt %, up to 55 wt %, up to 40 wt %, up to 35 wt % of carbon fibre. Said second composite material may include 30 to 70 wt % of said second polymeric material and 30 to 70 wt % of carbon fibre. In a preferred embodiment said second composite material comprises 60 to 80 wt % of polymeric material of formula I, preferably of formula I wherein t=1 and v=0, and 20 to 40 wt % of carbon fibre.

Said second composite material may include one or more further components. It may include up to 15 wt %, preferably up to 10 wt % of other components. An example of another component is an X-ray contrast material for example barium sulphate.

Said second composite material preferably includes only a single type of second polymeric material of formula I. It may also include only a single type of carbon fibre—e.g. only PAN-based; or only pitch-based, but not a mixture of two types.

Said first composite material and said second composite material preferably comprise the same polymeric material of formula I and preferably the same carbon fibre. Preferably, said first composite material and said second composite material have substantially the same composition.

The assembly of the first aspect may include one or more additional parts which may bear against said first and/or said second parts. Said one or more additional parts may be made from a said first composite material as described.

According to a second aspect of the invention, there is provided a kit for providing an assembly of said first aspect, the kit comprising:
(a) a first part as described according to said first aspect;
(b) a second part as described according to said first aspect;
   wherein said first part and said second part are cooperable to define an assembly wherein said first and second parts bear against one another.

Said first part and said second part may have any feature of the first part and the second part of the first aspect mutatis mutandis.

According to a third aspect of the invention, there is provides a package, which is preferably substantially sterile, which comprises an assembly or kit according to the first or second aspects respectively.

According to a fourth aspect, there is provided a method of manufacturing a first part and a second part as described according to the first and second aspects, the method comprising forming respective bearing surfaces of said first and second parts from a first composite material and a second composite material respectively.

The method may comprise making one or both of said parts substantially entirely from said first or second composite materials; or the method may comprise forming one or both bearing surfaces (but not the entirety) of said first and second parts out of said first and/or second composite materials.

According to a fifth aspect of the invention, there is provided a method of making an assembly according to the first aspect, the method comprising:
(a) selecting a first part as described according to the first aspect;
(b) selecting a second part as described according to the first aspect; and
(c) contacting the first and second parts so that the parts bear against one another and define said assembly.

According to a sixth aspect of the invention, there is provided the use of a first part as described according to the first aspect and a second part as described according to the first aspect in the manufacture of an assembly which comprises said first and second parts bearing against one another for implantation into the human body, for example to replace a structural element of the body.

Any feature of any aspect of the invention or embodiment described herein may be combined with any other feature of any aspect of an invention or embodiment described herein mutatis mutandis.

Specific embodiments of the invention will now be described by way of example.

The following materials are referred to hereinafter:

PEEK-OPTIMA LT1—Long term implantable grade polyetheretherketone with a melt viscosity of approximately 0.45 kNsm⁻², obtainable from Invibio Limited, UK.

CFR-PEEK-LT1—Implant grade polyetheretherketone containing 30% by weight PAN based carbon fibres, obtained from Invibio Limited, UK.

Acetal refers to poly(oxymethylene).

UHMWPE—refers to Ultra High Molecular Weight Polyethylene obtained from DuPuy Orthopaedics.

Pin-on-plate testing was used to assess materials. The pins and plates were made according to the general procedures described in Example 1 and 2 and tested using the general procedure described in Example 3.

### EXAMPLE 1

### General Procedure for Making Pins

All pins were machined from injection moulded plaques. All plaques were produced using standard conditions, for example those described in general literature available from Invibio Limited. The machined pins were polished to give a surface roughness (Sa) of approximately 1 micron. All pins were cleaned in aqueous ethanol and demineralised water and annealed using a general annealing protocol for example as described in general literature available from Invibio Ltd. All pins were machined such that any fibre alignment caused by the direction of polymer flow would be parallel with the reciprocating motion. Unless specified, all pins were gamma sterilised with an irradiation dose of 50 kGy.

### EXAMPLE 2

### General Procedure for Making Plates

All plates were machined from injection moulded plaques. All plaques were produced using standard conditions. The machined plates were machined to maintain the injection moulded surface finish (Sa of approximately 0.1 micron). All plates were cleaned in aqueous ethanol and demineralised water and annealed using a general annealing protocol. All plates were machined such that any fibre alignment caused by the direction of polymer flow would be parallel with the reciprocating motion.

### EXAMPLE 3

### General Procedure for Testing Materials

A pin-on-plate machine was used. The machine was a four station pin-on-plate machine which applied both reciprocation and rotational motion. The reciprocation was applied by a sledge moving along two fixed parallel hardened steel bars and a heated bed, lubricant tray and plate holder were positioned on top of this sledge. The rotational motion was applied to each pin using a small motor. The cycle frequencies of both the reciprocation and the rotation was set at approximately 1 Hz. The plate holder consisted of four wells into which the plate specimens were clamped. A lubricant was contained within the lubricant tray and heated to a temperature of 37° C. by resistors within the bed. This was controlled by a thermocouple. A load (either of 20 N or 40 N) as applied to each station via a lever arm mechanism. A lubricant level sensor made from platinum wire was attached to the lubricant tray to allow the lubricant to be maintained at an almost constant level. This was topped up from a reservoir of distilled water. An electronic counter was connected to the reciprocating sledge. Stroke length was set to 25 mm. A cover was placed over the entire rig to prevent dust contamination from the atmosphere.

The lubricant used was 24.5% bovine serum (protein content: 15 gl⁻¹) with 0.2% sodium azide added to retard the growth of bacteria and 20 mM EDTA to prevent calcium deposition.

The wear was assessed gravimetrically. At least twice a week (approx. 0.25 million cycles) the machine was stopped to allow for cleaning and weighing of the samples. Any excess lubricant was cleaned from the lubricant baths and the pins and plates removed. The samples were then cleaned and dried using a predetermined and consistent protocol. The pins and plates were then weighed three times on a balance (accurate to 0.1 mg) and an average weight recorded. Control specimens were used to take account of the lubricant absorption of both the pins and plates during the test duration. The machine was then reassembled and the lubricant refreshed. The wear tests were performed up to two million cycles.

Vacuum oven drying tests were also performed both before and after the wear tests in an attempt to get the ‘true’ weight loss of these materials and compare this to the standard weight loss measurements.

The wear volumes were plotted against sliding distance and the gradient of the line through the data (determined by linear regression analysis) provided the wear rate. The wear rate was then divided by the load and sliding distance to determine the wear factor, k (mm³N⁻¹m⁻¹).

### EXAMPLES 4 TO 12

Using the procedures described in Examples 1 and 2 pins and plates were made and combinations tested under specified loads, using the general procedure described in Example 3. A summary of materials used, the load applied and calculated wear factors is provided in Table 1. Table 2 describes volumetric wear for selected example.

TABLE 1

| Example | | Plate | | Wear factors (mm³ | | |
|---|---|---|---|---|---|---|
| No. | Pin material | material | Load (N) | N⁻¹ × 10⁻⁶ | | |
| 4 | Acetal | UHMWPE | 40N | 1.373 | 2.746 | 4.119 |
| 5 | UHMWPE | Acetal | 40N | 2.393 | 1.701 | 4.094 |
| 6 | UHMWPE | PEEK-OPTIMA | 40N | 5.431 | 0.529 | 5.960 |
| | | LT1 (Non- | | | | |
| | | Sterilised) | | | | |
| 7 | PEEK-OPTIMA | UHMWPE | 40N | 0.162 | 4.163 | 4.325 |
| | LT1 Non- | | | | | |
| | Sterilised. | | | | | |
| 8 | PEEK-OPTIMA | PEEK-OPTIMA | 40 | 2.34 | 2.33 | 4.67 |
| | LT1 non- | LT1 non- | | | | |
| | sterilised | sterilised | | | | |
| 9 | PEEK-OPTIMA | PEEK-OPTIMA | 40 | 1.92 | 2.58 | 4.50 |
| | LT1 | LT1 | | | | |
| 10 | PEEK-OPTIMA | PEEK-OPTIMA | 20 | 2.30 | 3.56 | 5.86 |
| | LT1 | LT1 | | | | |
| 11 | PEEK-OPTIMA | PEEK-OPTIMA | 40 | 0.07 | 0.27 | 0.34 |
| | LT1 with 30% | LT1 with 30% | | | | |
| | PAN Carbon | PAN Carbon | | | | |
| | Fibres | Fibres | | | | |
| 12 | PEEK-OPTIMA | PEEK-OPTIMA | 20 | 0.36 | 0.53 | 0.89 |
| | LT1 with 30% | LT1 with 30% | | | | |
| | PAN Carbon | PAN Carbon | | | | |
| | Fibres | Fibres | | | | |
| 13 | UHMWPE Gamma | Stainless | 40N | 1.1 | 0 | 1.1 |
| | Sterilised | Steel | | | | |
| 14 | High Carbon | High Carbon | 40N | 0.78 | 0.06 | 0.84 |
| | CoCrMo | CoCrMo | | | | |

TABLE 2

| | | | | Volumetric Wear | | |
|---|---|---|---|---|---|---|
| Example | | Plate | | (mm³/million cycles) | | |
| No. | Pin material | material | Load (N) | Pin | Plate | Total |
| 10 | PEEK-OPTIMA | PEEK-OPTIMA | 20 | 2.3 | 3.68 | 5.98 |
| | LT1 | LT1 | | | | |
| 9 | PEEK-OPTIMA | PEEK-OPTIMA | 40 | 3.59 | 4.97 | 8.56 |
| | LT1 | LT1 | | | | |
| 12 | PEEK-OPTIMA | PEEK-OPTIMA | 20 | 0.35 | 0.49 | 0.84 |
| | LT1 with 30% | LT1 with 30% | | | | |
| | PAN Carbon | PAN Carbon | | | | |
| | Fibres | Fibres | | | | |
| 11 | PEEK-OPTIMA | PEEK-OPTIMA | 40 | 0.15 | 0.53 | 0.68 |
| | LT1 with 30% | LT1 with 30% | | | | |
| | PAN Carbon | PAN Carbon | | | | |
| | Fibres | Fibres | | | | |

### EXAMPLE 13

By processes analogous to the processes described above, the wear performance of a composite comprising PEEK-OPTIMA LT1 and PAN carbon fibre pins and plates bearing against one other was compared to the wear performance of a composite comprising PEEK-OPTIMA LT1 and pitch-based carbon fibre pins and plates bearing against each other.

After 5 million cycles the results are as follows:

| Wear couple | | Total wear factor mm³N⁻¹mm⁻¹ × 10⁻⁶ |
|---|---|---|
| CFR-PEEK | CFR-PEEK | 0.25 |
| (PAN) | (PAN) | |
| CFR-PEEK | CRF-PEEK | 0.92 |
| (Pitch) | (Pitch) | |

### Results and Discussion

Referring to Table 1, it can be seen that the total wear of the non-sterilised PEEK coupling is similar to the total wear of the sterilised PEEK coupling.

The carbon fibre-PEEK samples articulating against the same material (Examples 11 and 12) gave lower wear than the all-PEEK components (Examples 8 to 10) and indeed the lowest wear for any of the all polymeric wear couples tested. The total wear factors for the test using a 40 N load were thirteen times lower for the carbon fibre-PEEK material (Examples 11 and 12) than the PEEK samples (Examples 7 to 10) and for the test using a 20 N load, they were six times lower.

The wear factors for PEEK-OPTIMA LT1 containing 30% PAN carbon fibres articulating against the same material (example 11) has a lower wear factor than that of traditionally used successful bearing couples used in medical implants. When compared with UHMWPE articulating against metal (example 13) a greater than 60% reduction in wear factor was observed for PEEK-OPTIMA LT1 containing 30% PAN carbon fibres articulating against the same material. When compared with a metal on metal wear couple (example 14) a greater than 40% reduction in wear factor was observed for PEEK-OPTIMA LT1 containing 30% PAN carbon fibres articulating against the same material.

Referring to Table 2, the volumetric wear of PEEK-OPTIMA LT1 articulating against the same material, unsurprisingly showed that under a 40 N load higher actual wear rates were observed than when under a 20 N load. This is expected as the wear rate should increase with an increase in the applied load see T A Stolarski, Wear 1992, 158, 71-78. “Tribology of polyetheretherketone”; S M Hosseini and T A Stolarski, Journal of Applied Polymer Science, 1992, 45, 2021-2030, “Morphology of Polymer Wear Debris Resulting from Different Contact Conditions”; M Q Zhang, Z P Lu and K Friedrich, Tribology International 1997, 30, 103-111; Z P Lu and K Friedrich, Wear 1995, 181-183, 624-631, “On sliding friction and wear of PEEK and its composites”; T J Joyce, H E Ash and A Unsworth, Proc. Instn. Mech Engineers 1996, 210, 11, “The wear of cross-linked polyethylene against itself”; T J Joyce and A Unsworth, Proc. Instn. Mech Engineers 1996, 210, 297, “A comparison of the wear of cross-linked polyethylene against itself with the wear of ultrahigh molecular weight polyethylene against itself”. For the PEEK samples the volumetric wear rate with the 40 N load was 8.56 mm³/million cycles and for the 20 N load this was 5.98 mm³/million cycles.

However, surprisingly, the carbon fibre-PEEK components demonstrated a lower volumetric wear rate for the 40 N load (0.68 mm³/million cycles), than for the same wear couple tested under 20 N loads (0.84 mm³/million cycles).

Referring to Example 13, it appears that a wear couple comprising PEEK and PAN-based carbon fibres exhibits lower wear compared to a couple comprising Pitch-based fibres.

It should now be appreciated that, in particular, the composite materials described may advantageously be used in bearing applications—they have low wear rates and the material may advantageously be used for bearing surfaces for reconstructive joints or other parts. It should also be noted that these materials demonstrate an improvement in wear performance at increased loads and therefore there may be benefits to using these materials in high load applications such as total knee joints.

In comparison with metal or ceramic components these materials can be manufactured by a lower cost and more efficient manufacturing route such as injection moulding. There may be additional benefits in using these lower modulus materials compared with metals or ceramics, which can cause stress shielding and subsequent bone resorption.

Other advantages of the materials described are that they are less brittle than ceramics; and use of the materials avoids the production of metallic wear debris and the associated health risk of metal ions being released into the body (see for example R Michel, J Hofman, F Loer and J Zilkens “Trace element burdening of human tissues due to the corrosion of hip joint prostheses made of cobalt chrome molybdenum” and Arch. Orthop. and Traumat. Surg. 1984, 103, 85-95; and T Visuri, E Pukkala, P Paavolainen, P Pulkkinen, E B Riska, Clin Orthop 1996; 329:S280-289, wherein it is described how in patients who had a metal on metal total hip replacement the total risk of cancer was found to be 1.23 times higher than that experienced by patients with PE on metal total hip replacements).

Further advantages of the composite materials described include lower weight than metals or ceramics; and improved mechanical properties compared with UHMWPE thereby allowing thinner parts, a greater degree of motion and design flexibility.

1. An assembly comprising:
(a) a first part which comprises a first composite material which includes a first polymeric material and carbon fibre, wherein said first polymeric material includes a repeat unit of formula 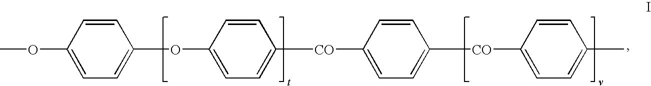
and;
(b) a second part which comprises a second composite material which includes a second polymeric material and carbon fibre, wherein said second polymeric material includes a repeat unit of formula 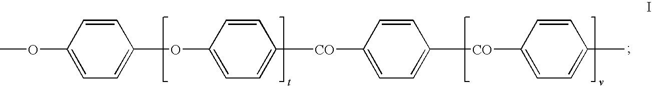
wherein said first and second parts bear against one another.

2. An assembly according to claim 1, wherein said first part and said second part are movable relative to one another.

3. An assembly according to claim 1, wherein said first and second parts are lubricated in use.

4. An assembly according to claim 2, wherein said assembly is for implantation in a human body so as to replace a structural element of the human body.

5. An assembly according to claim 1, said assembly being for use in or around the spine; or for use in an artificial joint.

6. An assembly according to claim 1, wherein one of said first or second parts comprises a male element and the other of said first or second parts comprises a female element wherein said male and female elements bear against one another.

7. An assembly according to claim 1, wherein a bearing surface of said first part which comprises said first composite material contacts a bearing surface of said second part which comprises said second composite material.

8. An assembly according to claim 1, wherein:
said first part is made substantially entirely from said first composite material; or said first part comprises a material other than said first composite material but a bearing surface of said first part is defined by said composite material; and
said second part is made substantially entirely from said second composite material; or said second part comprises a material other than said second composite material but a bearing surface of such a second part is defined by said second composite material.

9. An assembly according to claim 4, wherein one of said first or second parts of the assembly defines a head and the other part defines a socket within which the head is pivotable.

10. An assembly according to claim 1, wherein said assembly is for a hip replacement.

11. An assembly according to claim 1, wherein said first polymeric material is a general formula of I, wherein t=1 and v=0.

12. An assembly according to claim 1, wherein said first polymeric material includes at least 60 mole % of repeat units of formula I.

13. An assembly according to claim 1, wherein said first polymeric material consists essentially of a repeat unit of formula I wherein t=1 and v=0 and said second polymeric material consists essentially of a repeat unit of formula I wherein t=1 and v=0.

14. An assembly according to claim 1, wherein said first polymeric material and said second polymeric material are the same.

15. An assembly according to claim 1, wherein said first composite material includes at least 30 wt % of said first polymeric material and up to 70 wt % of carbon fibres.

16. An assembly according to claim 1, wherein said first composite material comprises 60 to 80 wt % of polymeric material of formula I and 20 to 40 wt % of carbon fibre.

17. An assembly according to claim 1, wherein said second composite material comprises 60 to 80 wt % of polymeric material of formula I and 20 to 40 wt % of carbon fibre.

18. An assembly according to claim 1, wherein said first part comprises a first composite material comprising said first polymeric material and PAN-based carbon fibres and said second part comprises said second composite material comprising said second polymeric material and PAN-based carbon fibres.

19. A kit for providing an assembly of claim 1, the kit comprising:
(a) a first part as described according to claim 1; and
(b) a second part as described according to claim 1;
wherein said first part and said second part are cooperable to define an assembly wherein said first and second parts bear against one another.

20. A package comprising an assembly according to claim 1.

21. A method of manufacturing a first part and a second part as described according to claim 1, the method comprising forming respective bearing surfaces of said first and second parts from a first composite material and a second composite material respectively.

22. A method of making an assembly according to claim 1, the method comprising:
(a) selecting a first part as described in claim 1;
(b) selecting a second part as described in claim 1; and
(c) contacting the first and second parts so that the parts bear against one another and define said assembly.

23. The use of a first part according to claim 1 and a second part according to claim 1 in the manufacture of an assembly which comprises said first and second part bearing against one another for implantation into the human body.

24. A package comprising a kit according to claim 19.

25. A method of providing a reconstructive joint in a human body, the method comprising implanting into the human body an assembly as claimed in claim 1.

26. A reconstructive joint for a human body, the joint comprising a first part and a second part which bear against one another, wherein said first part comprises a first composite material which comprises 20 to 40 wt % of carbon fibre and 60 to 80 wt % of polymeric material of formula 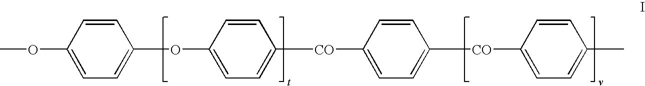
wherein t and v independently represent 0 or 1; and
wherein said second part comprises a second composite material which comprises 20 to 40 wt % of carbon fibre and 60 to 80 wt % of polymeric material of formula 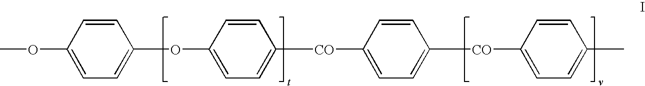
wherein t and v independently represent 0 or 1.

27. A joint according to claim 26, wherein said first part and said second part include PAN-based carbon fibres.
